(19) United States
(12) Patent Application Publication
(10) Pub. No.: US 2013/0217976 A1
(43) Pub. Date: Aug. 22, 2013

(54) Method for Predicting Quality of Life After Medical and Surgical Treatment

(71) Applicants: B. Todd Heniford, Charlotte, NC (US); John I. Shipp, Atlantic Beach, FL (US)

(72) Inventors: B. Todd Heniford, Charlotte, NC (US); John I. Shipp, Atlantic Beach, FL (US)

(21) Appl. No.: 13/774,993

(22) Filed: Feb. 22, 2013

Related U.S. Application Data
(60) Provisional application No. 61/601,797, filed on Feb. 22, 2012.

(51) Int. Cl. A61B 5/00

(52) U.S. Cl. CPC A61B 5/4848
USPC 600/300

(57) ABSTRACT

A method of determining the risk of performing a procedure on a patient is provided. An algorithm is presented that allows the determination of the risks associated with a procedure and a determination can then be made as to whether the procedure should be done.

### BACKGROUND OF THE INVENTION

This application claims the benefit of application Ser. No. 61/601,797, filed on Feb. 22, 2012, which is hereby incorporated by reference in its entirety.

### FIELD OF THE INVENTION

The present invention relates generally to a method of determining whether to perform a medical procedure based on the predictive outcome of the medical procedure and the effect on the patient after the medical procedure.

### TECHNICAL BACKGROUND

Management of various diseases or surgical problems has allowed patients a cure, partial treatment or palliation of their disease, injury, or acquired or genetic medical or surgical conditions. These therapies or the avoidance of these therapies impact the quality of life (“QOL”) of patients.

By gathering data from the patient in the pre-treatment arena (including QOL data, personal, medical and surgical histories, physical examination information, laboratory, organ and physical function, radiographic data, and other health, personal, psychological, and related data), acquiring information during and after any and all forms of medical and surgical therapy, and by determining medical, surgical, functional and psychological outcomes and QOL of the patient, these data can be studied and analyzed to predict QOL outcomes. Using statistical functions such a univariate and multivariate logistic regression model, Fisher's exact, Chi-square test, principal component analysis, and/or forward stepwise logistic regression, among others, the outcomes of therapies or the lack of therapy can be predicted via a mathematical equation or algorithm including the QOL. The more important variables will be weighted more significantly in this equation to allow for greater influence in the outcomes measure.

Thus, a predictive model has been developed to determine based on gathered information, whether a particular procedure will affect the quality of life of a patient.

### SUMMARY OF THE INVENTION

Disclosed herein is a method for determining the risk of performing a procedure on a patient that includes the steps of taking a patient history from the patient who is a candidate for the procedure, performing a physical examination of the patient, determining the procedure to be performed, and predicting the quality of life of the patient after the procedure based on the patient history and the physical examination.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description of the present embodiments of the invention are intended to provide an overview or framework for understanding the nature and character of the invention as it is claimed. The accompanying drawings are included to provide a further understanding of the invention and are incorporated into and constitute a part of this specification. The drawings illustrate various embodiments of the invention and, together with the description, serve to explain the principles and operations of the invention.

### BRIEF DESCRIPTION OF THE DRAWINGS

FIG. 1 is a table illustrating the demographics of the population used to create the predictive model according to one embodiment of the present invention;

FIG. 2 is a table illustrating the effect of two variables on the probability of chronic pain after a procedure;

FIG. 3 is a table illustrating the analysis of chronic pain confounders; and

FIG. 4 is a table illustrating the odds ratios of independent predictors of chronic pain.

### DETAILED DESCRIPTION OF THE PREFERRED EMBODIMENTS

Reference will now be made in detail to the present preferred embodiment(s) of the invention and with reference to the information illustrated in the accompanying drawings. Whenever possible, the same reference numerals will be used throughout the drawings to refer to the same or like parts.

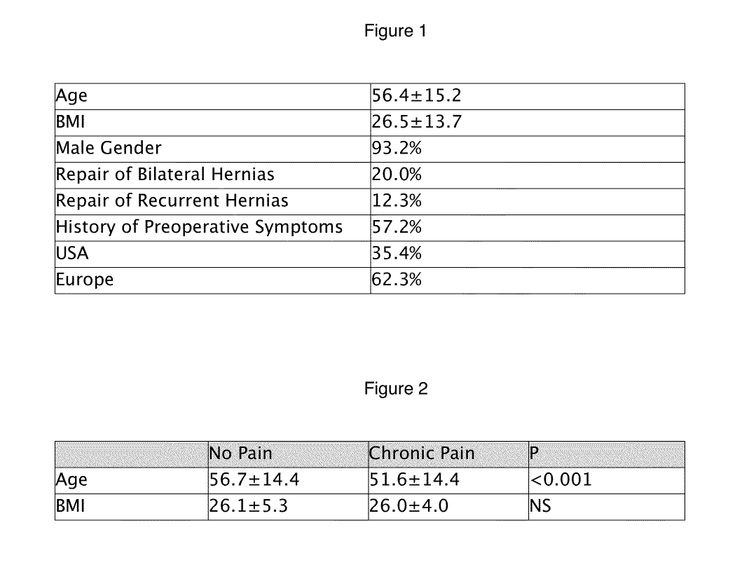

As one example, inguinal hernia repair is one of the most common operations performed in the United States and throughout the world. However, patients undergoing repair have a 7-40% incidence of chronic pain lasting months or indefinitely. To better understand and predict QOL after inguinal hernia repair, patients undergoing inguinal hernia repair in a prospective trial from 2007 to 2011 were included in an analysis. Initially, peri-operative factors predictive of chronic discomfort or pain long-term were identified. As well, in this and other studies, patients with potential postoperative confounders can be excluded. Carolinas Comfort Scale (CCS), a treatment, QOL tool was used to compare postoperative symptoms outcomes at 1 year follow-up. A score of 2 or higher (mild but bothersome to severe) was considered symptomatic. Multivariate logistic regression model was used to calculate the adjusted odds ratios of factors contributing to chronic symptoms and develop a predictive mathematical algorithm. Of the 2,497 subjects, 1,718 patients (75.5%) were seen at one-year follow-up. A random sample of 80% of these patients was used for a chronic pain predictive model. The remaining 20% were used for predictive model validation. FIG. 1 illustrates the demographics of the 1,718 patients who were evaluated at the one-year follow-up visit.

Using the information from these patients, a univariate analysis was performed on the data from these patients to identify potential confounders of chronic pain at the one-year visit by comparing symptomatic to asymptomatic patients. The table in FIG. 2 illustrates the univariate analysis of the chronic pain confounders−age of the patients and each patient's BMI (body mass index).

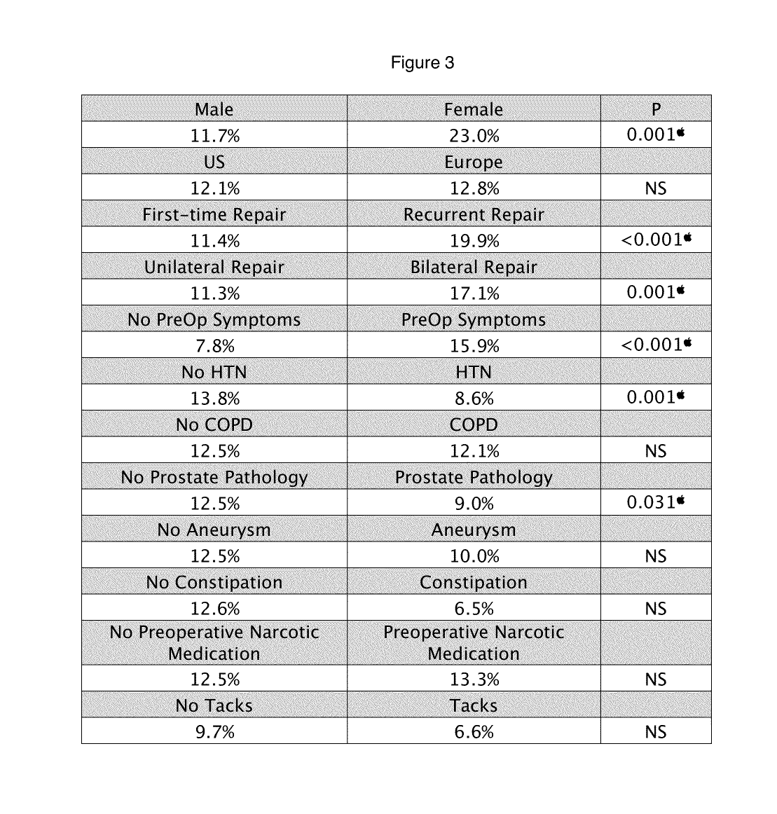

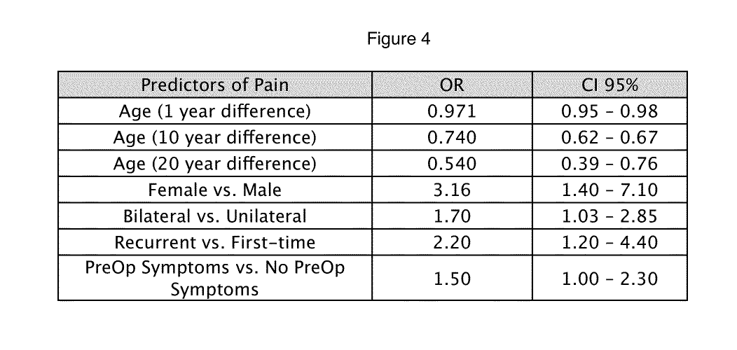

The table in FIG. 3 illustrates the univariate analysis of a variety of chronic pain confounders. These include gender, country (US or Europe), repair status (first time or recurrent−second or more), whether it is a unilateral or bilateral repair, whether there were pre-operative symptoms (e.g., pain), hypertension, presence of Chronic obstructive pulmonary disease (COPD), prostate pathology, presence or absence of an aneurysm, constipation, pre-operative narcotic medication, and use of tacks in the prior repair. It can be seen from the table that gender, repair status, whether it is a unilateral or bilateral repair, whether there were pre-operative symptoms (e.g., pain), and prostate pathology were statistically significant. Using a logistic regression, it has been demonstrated that gender, younger age, recurrent repair, bilateral repair and the presence of preoperative pain were identified as independent predictors of chronic pain. The table in FIG. 4 illustrates the odds ration associated with each of these independent confounders.

Principal component analysis was performed on all pre-operative CCS responses (pain+movement questions). Forward stepwise logistic regression (FSLR) was then performed. Pertinent clinical variables and significant principle components were included as potential explanatory/predictor variables. The result was a predictive algorithm:

P=0.1190−(0.0306*age)+(0.5746*gender)−(0.3955*primary/recurrent)+(0.2343*bilateral/unilateral)+(0.1114*prin1),

where age is the age of the patient in years, gender is 1 for female and −1 for male, primary/recurrent is 1 for primary and −1 for recurrent procedure, and bilateral/unilateral is 1 for bilateral and −1 for unilateral, and prin1 is principal component analysis using preoperative pain and movement limitations.

The sensitivity and specificity of this predictive algorithm were 70% and 65% respectively. Eighty percent of the original group of patients was utilized to develop the algorithm or sophisticated mathematical equation above. The remaining 20% were then tested for predictive model validation and confirmed its predictive value with sensitivity and specificity of this predictive algorithm remaining between 60% and 70%.

As a result, a prediction can be made about the patient's response to the procedure that is being suggested. After taking a patient history that covers the confounders noted above, performing a physical examination of the

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

1. A method of determining the risk of performing a procedure on a patient comprising the steps of:
taking a patient history from the patient who is a candidate for the procedure;
performing a physical examination of the patient;
determining the procedure to be performed; and
predicting the quality of life of the patient after the procedure based on the patient history and the physical examination.

2. The method according to claim 1, wherein the procedure is a medical procedure.

3. The method according to claim 2, wherein the medical procedure is a hernia repair procedure.

4. The method according to claim 1, wherein the taking a patient history includes the age, BMI, gender, prior procedures of the same kind; country

5. The method according to claim 1, further comprising performing the procedure based on the predicted quality of life.

6. The method according to claim 1, wherein the step of predicting the quality of life comprises evaluating the potential for pain post-procedure.

7. The method according to claim 6, wherein the step of evaluating the potential for pain is determined using the formula P=0.1190−(0.0306*age)+(0.5746*gender)−(0.3955*primary/recurrent)+(0.2343*bilateral/unilateral)+(0.1114*prin1), where age is the age of the patient in years, gender is 1 for female and −1 for male, primary/recurrent is 1 for primary and −1 for recurrent procedure, and bilateral/unilateral is 1 for bilateral and −1 for unilateral, and prin1 is the principal component analysis using preoperative pain and movement limitations.

8. The method according to claim 7, wherein the patient will not experience ongoing pain if P is less than −2.4369 and will and will experience ongoing pain if P is greater than −2.4369.
